(19) United States
(12) Patent Application Publication
(10) Pub. No.: US 2007/0160688 A1
(43) Pub. Date: Jul. 12, 2007

(54) Use of flavonoids to increase blood serum levels of dehydroepiandrosterone

(76) Inventor: Eric Marchewitz, Howell, MI (US)

Correspondence Address: Miller, Canfield, Paddock and Stone P.L.C.;c/o Robert Kelley Roth, Suite 2500, 150 West Jefferson Ave., Detroit, MI, 48226, US

(21) Appl. No.: 11/652,294

(22) Filed: Jan. 10, 2007

Related U.S. Application Data
(60) Provisional application No. 60/757,779, filed on Jan. 10, 2006.

(51) Int. Cl. A61K 36/725; A61K 36/539; A61K 36/906; A61K 36/48; A61K 36/37; A61K 31/7048; A61K 31/353; A61K 36/575

(52) U.S. Cl. 424/725; 424/756; 424/757; 424/759; 424/741; 514/456; 514/27

(57) ABSTRACT

A method is disclosed for increasing blood serum levels of dehydroepiandrosterone by administering the combination of one of a flavonoid, isoflavonoid, coumarin and a flavonolignan; dehydroepiandrosterone; and a plant extract.

### RELATED APPLICATIONS

This application claims priority benefit of U.S. provisional patent application No. 60/757,779, filed on Jan. 10, 2006.

### FIELD OF THE INVENTION

This invention relates to use of certain flavonoids to maintain and increase blood serum levels of dehydroepiandrosterone (3-beta-hydroxy-5-androst-1-en-17-one or simply “DHEA”) and decrease production of undesirable metabolites of DHEA to enhance physical performance, and more particularly to the use of flavonoids for anti-aging benefits, decreasing body weight, reducing adipose tissue, and increasing endurance.

### BACKGROUND OF THE INVENTION

The adrenal gland produces many steroid hormones. These steroid hormones play a major role in many body processes including, for example, skeletal muscle growth, red blood cell production (erythropoiesis), regulation of glucose and insulin levels and cellular aging. The steroids produced by the adrenal gland can be divided into three groups: glucocorticoids, which influence carbohydrate metabolism; mineralocorticoids, which control electrolyte and water balance; and sex steroid hormones. Glucocorticoids such as cortisol regulate catabolism of skeletal muscle proteins into amino acids. These amino acids are then transported to the liver and converted into glucose during gluconeogenesis. Excessive amounts of glucocorticoids can result in higher blood glucose and insulin levels and can contribute to increased body fat and type II diabetes. Glucocorticoids are also known to play a role in the aging process by increasing cellular and mitochondrial breakdown.

The second group of adrenal steroids, the mineralocorticoids such as aldosterone help the body to retain sodium and water. Excesses of mineralocorticoids can result in hypertension and cardiovascular disease.

The third group of adrenal steroids include androgens and DHEA. Adrenal androgens oppose the actions of glucocorticoids and result in skeletal muscle anabolism (the opposite action of catabolism), reductions in blood glucose and insulin levels, reduction in body fat, and are believed to decrease the rate of cellular aging and increased red blood cell production. DHEA production by the adrenal glands is known to decline markedly as aging progresses.

With normal younger adults, all three groups of adrenal steroids are produced in healthy amounts. However, as people age, less DHEA is produced resulting in relatively greater amounts of glucocorticoids and mineralocorticoids.

DHEA and DHEA derivative supplementation is believed be useful in treatment of aging and obesity and to stimulate erythropoiesis and skeletal muscle anabolism. In addition, administering supplemental DHEA and DHEA derivatives can help restore the balance of adrenal steroids.

DHEA is commonly used as a dietary supplement. Unfortunately, DHEA is rapidly metabolized by liver enzymes referred to as sulfotransferases. Sulfotransferases rapidly convert the much of the supplementary DHEA into DHEA sulfate, which is quickly excreted from the body and is not effective as an anti-aging, muscle-building or fat reduction compound. In addition, DHEA sulfate does not restore the balance of the adrenal steroids discussed above. As a result, frequent and larger doses of DHEA must be taken.

DHEA is also metabolized in the body to one of several compounds including, for example, etiocholanolone(5-beta-androstan-3-alpha-ol-17-one), beta etiocholanolone(5-beta-androstan-3-beta-ol-17-one), androsterone(5-alpha-androstan-3-alpha-ol-17-one), epiandrosterone(5-alpha-androstan-3-beta-ol-17-one), 7-keto-DHEA, 7-alpha-hydroxy-DHEA, 7-beta-hydroxy-DHEA, androstenedione, estrone and estradiol. There is great individual variability in the metabolism of oral DHEA. The DHEA metabolites estrone and estradiol can result in negative estrogenic side effects for males including growth of male breast tissue, known as gynecomastia. Some individuals have poor bioavailability of DHEA as a result of sulfation in the liver, and large doses must be taken to elicit any desired effects. These increased doses of DHEA can result in increased conversion to estrone and estradiol, with resulting negative side effects.

It would be desirable to provide compounds which can be used to help provide the beneficial effects of high DHEA levels in the body for extended periods of time, yet reduce the undesired DHEA side effects discussed above.

### SUMMARY OF THE INVENTION

In accordance with a first aspect, a method is disclosed for maintaining and increasing blood serum levels of dehydroepiandrosterone and decrease production of undesirable metabolites of DHEA to enhance physical performance by administering the combination of a flavonoid, isoflavonoid, coumarin or a flavonolignan, dehydroepiandrosterone and a plant extract.

From the foregoing disclosure and the following more detailed description of various preferred embodiments it will be apparent to those skilled in the art that the present invention provides a significant advance in the methods of administering anti-aging compounds, for decreasing body weight and reducing adipose tissue, and increasing endurance. Additional features and advantages of various preferred embodiments will be better understood in view of the detailed description provided below.

### DETAILED DESCRIPTION OF CERTAIN PREFERRED EMBODIMENTS

It will be apparent to those skilled in the art, that is, to those who have knowledge or experience in this area of technology that many variations are possible for the method of increasing blood serum levels of DHEA for enhancing physical performance. The following detailed discussion of various alternative and preferred features and embodiments will illustrate the general principles of the invention with reference to improved methods of enhancing physical performance by administering flavonoids as orally available dietary supplements. Other embodiments suitable for other applications will be apparent to those skilled in the art given the benefit of this disclosure.

This invention discloses the method of administering flavonoids, isoflavonoids, coumarins and flavonolignans and/or plant extracts and optionally in combination with DHEA or its derivatives for the purpose of decreasing DHEA conversion to DHEA sulfates, decreasing, for example, DHEA glucuronide conjugates androstenedione and androgenic or estrogenic metabolites, and thereby increasing blood serum levels of DHEA.

As used herein, a derivative of a compound refers to a species having a chemical structure that is similar to the compound, yet containing a chemical group not present in the compound and/or deficient of a chemical group that is present in the compound. The substance to which the derivative is compared is known as the “parent” substance.

Certain flavonoids(2-phenyl-1,4-benzopyronesand their derivatives), coumarins, (1,2-Benzopyrones and their derivatives), isoflavonoids(3-phenyl-1,4-benzopyrones and their derivatives) and flavonolignans (combination of a flavonoid and a lignane) can as act as substrates and/or inhibitors of the sulfotransferases that convert DHEA to DHEA sulfate. For example, the flavonoid quercetin, or the isoflavonoids genistein and daidzein, reproduced below, may be used in combination with a plant extract to increase DHEA levels.

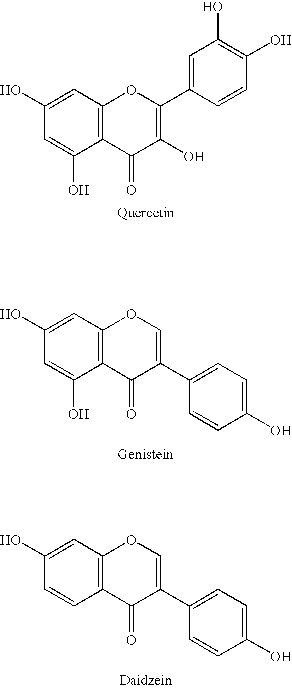

All of the naturally occurring compounds disclosed herein would preferably be administered orally mixed with solid or liquid carriers in appropriate unit doses. When taken with a plant extract and with DHEA the result is the reduction of the sulfation of the DHEA in the liver. This method of inhibiting sulfotransferase activity on DHEA increases DHEA blood serum levels as well as reducing androgenic and estrogenic activity in the body. More specifically, several enzymes involved in the breakdown of DHEA are inhibited, including sulfotransferase, 3-beta hydroxysteroid dehydrogenase, aromatase, 5-alpha reductase and glucuronsyltransferase. Also androgen and estrogen receptors may be blocked, increasing the duration of action and plasma concentration of DHEA and reducing estrogenic and/or androgenic action.

Suitable flavonoids for use herein include, for example, the following: Flavone; flavonone; Minimiflorin; 3-Hydroxyflavone; 5-Hydroxyflavone[Primuletin]; 6-Hydroxyflavone; 6-Hydroxyflavanone; 7-Hydroxyflavone; 3,4-Dihydroxyflavone; 3,6-Dihydroxyflavone; 3,7-Dihydroxyflavone; 5,7-Dihydroxyflavone[Chrysin]; 7,8-Dihydroxyflavone; 3,5,7-Trihydroxyflavone[Galangin]; 4,5,7-Trihydroxyflavone[Apigenin]; 4,5,7-Trihydroxyflavanone[Naringenin]; 5,6,7-Trihydroxyflavone[Baicalein]; 3,3,4,7-Tetrahydroxyflavone[Fisetin]; 3,4,5,7-Tetrahydroxyflavone[Kaempferol]; 3,4,5,7-Tetrahydroxyflavone[Luteolin]; 4-Methoxy-3,5,7-trihydroxyflavone[Hesperetin]; 3,4,7,8-Tetrahydroxyflavone; 2,3,4,5,7-Pentahydroxyflavone[Morin]; 3,3,4,5,7-Pentahydroxyflavone[Quercetin]; 3,4,5,7-tetrahydroxyflavone-3-rutinoside[Rutin]; 3,3,4,5,5,7-Hexahydroxyflavone[Myricetin]; (2R,3S)-2-(3,4-dihydroxyphenyl)-3,4-dihydro-1(2H)-benzopyran-3,5,7-triol[(+)Catechin]; (2S,3R)-2-(3,4-dihydroxyphenyl)-3,4-dihydro-1(2H)-benzopyran-3,5,7-triol[(−)Catechin]; (2S,3S)-2-(3,4-dihydroxyphenyl)-3,4-dihydro-1(2H)-benzopyran-3,5,7-triol[(+)Epicatechin]; (2R,3R)-2-(3,4-dihydroxyphenyl)-3,4-dihydro-1(2H)-benzopyran-3,5,7-triol[(−)Epicatechin].

Certain isoflavonoids can also act as inhibitors of 3-beta hydroxysteroid dehydrogenase. 3-beta hydroxysteroid dehydrogenase can convert DHEA to androstenedione. The inhibition of 3-beta hydroxysteroid dehydrogenase by isoflavonoids reduces the metabolism of DHEA thereby prolonging its duration of action. Certain isoflavonoids can also as act as substrates and/or inhibitors of the sulfotransferases that convert DHEA to DHEA sulfate. These isoflavonoids, including the soy isoflavones genistein and daidzein are sulfated by the liver sulfotransferases. When taken with DHEA the result is the reduction of the sulfation of the DHEA in the liver. This method of inhibiting sulfotransferase activity on DHEA increases the blood serum levels of DHEA. As a result the positive benefits of DHEA to the body lost through the sulfation process are reduced. Suitable isoflavonoids for use herein include, for example: Formononetin, Genistein, Equol, 3,4,7-Trihydroxyisoflavone, Biochanin A or Daidzein.

Certain coumarins can act as substrates for glucuronidases that convert DHEA to DHEA glucuronidate. Suitable coumarins for use herein include, for example Mammea A/BA or Mammea C/OA.

Suitable flavonolignans for use herein include, for example, silymarin, silybin A, silybin B, silybinin, dehydrosilybinin, silydianin and silychristin.

Certain plant extracts can be used in combination with the above mentioned compounds to help reduce the androgenic side effects of DHEA by interfering with either the metabolism of DHEA to androgenic metabolites or by interfering with the androgenic metabolites action at androgen receptors. Plant extracts such as angelica gigas, pygeum africanum, tripterygium wilfordii or scutellaria baicalensis advantageously reduce androgenic activity. The use of these plant extracts in combination with DHEA would reduce the likelihood of negative androgenic side effects. Suitable plant extracts for use herein also include, for example: croton roxburghii, zizyphus, jujube, coleus forkolii, achyranthes aspera, torilis japonica, artocarpus incisus, impatiens balsamina, alpinia officinarum, epilobium, magnolia, pueraria thumbergiana, scoparia dulcis, angelica gigas, pygeum africanum, tripterygium wilfordii and scutellaria baicalensis.

Flavonoids, isoflavonoids, coumarins or flavonolignans and plant extracts may be administered to an individual in combination with DHEA, 7-keto DHEA, 7-hydroxy DHEA or other derivatives of DHEA in order to increase the beneficial effects of these compounds. The preferred amount of the active ingredient that is to be administered would depend on various factors such as the age and weight of the user. An effective oral daily dosage of the described can comprise 50-2000 mg of DHEA daily, and most preferably about 100-250 mg of DHEA daily. A preferred embodiment might be to administer the oral dose as a soft gelatin capsule or oral liquid suspension, in two three or four divided doses per day (e.g, 50 to 400 mg of DHEA twice per day).

### EXAMPLE 1

Capsules. 25 kg of the compound DHEA is mixed with 5 kg of chrysin, and placed into 100,000 hard-gelatin capsules each with 250 mg of DHEA and 50 mg of Chrysin. Also silymarin may be added to decrease the glucuronidation of DHEA, prolonging its half-life. Silymarin is a flavonolignan mixture of silybin, isosilybin, silychristin and silydianin obtained from seeds of Silybum marianum.

### EXAMPLE 2

Capsules. 10 kg DHEA is mixed with 50 kg of croton roxburghii, and placed into 100,000 hard-gelatin capsules each with a content of 100 mg of DHEA and 500 mg of croton roxburghii.

From the foregoing disclosure and detailed description of certain preferred embodiments, it will be apparent that various modifications, additions and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method of increasing blood serum levels of dehydroepiandrosterone by administering the combination of one of a flavonoid, isoflavonoid, coumarin and a flavonolignan; one of DHEA and a derivative of DHEA; and a plant extract.

2. The method of claim 1 wherein the flavonoid is selected from the group of Flavone; flavonone; Minimiflorin; 3-Hydroxyflavone; 5-Hydroxyflavone; 6-Hydroxyflavone; 6-Hydroxyflavanone; 7-Hydroxyflavone; 3,4-Dihydroxyflavone; 3,6-Dihydroxyflavone; 3,7-Dihydroxyflavone; 5,7-Dihydroxyflavone; 7,8-Dihydroxyflavone; 3,5,7-Trihydroxyflavone; 4,5,7-Trihydroxyflavone; 4,5,7-Trihydroxyflavanone; 5,6,7-Trihydroxyflavone; 3,3,4,7-Tetrahydroxyflavone; 3,4,5,7-Tetrahydroxyflavone; 3,4,5,7-Tetrahydroxyflavone; 4-Methoxy-3,5,7-trihydroxyflavone; 3,4,7,8-Tetrahydroxyflavone; 2,3,4,5,7-Pentahydroxyflavone; 3,3,4,5,7-Pentahydroxyflavone; 3,4,5,7-tetrahydroxyflavone-3-rutinoside; 3,3,4,5,5,7-Hexahydroxyflavone; (2R,3S)-2-(3,4-dihydroxyphenyl)-3,4-dihydro-1(2H)-benzopyran-3,5,7-triol; (2S ,3R)-2-(3,4-dihydroxyphenyl)-3,4-dihydro-1(2H)-benzopyran-3,5,7-triol; (2S,3S)-2-(3,4-dihydroxyphenyl)-3,4-dihydro-1(2H)-benzopyran-3,5,7-triol; and (2R,3R)-2-(3,4-dihydroxyphenyl)-3,4-dihydro-1(2H )-benzopyran-3,5,7-triol.

3. The method of claim 1 wherein the isoflavonoid is selected from the group of Formononetin, Genistein, Equol, 3,4,7-Trihydroxyisoflavone, Biochanin A or Daidzein.

4. The method of claim 1 wherein the coumarin is selected from the group of Mammea A/BA or Mammea C/OA.

5. The method of claim 1 wherein the flavonolignan is selected from the group of silymarin, silybin A, silybin B, silybinin, dehydrosilybinin, silydianin and silychristin.

6. The method of claim 1 wherein the plant extract is selected from the group of croton roxburghii, zizyphus, jujube, coleus forkolii, achyranthes aspera, torilis japonica, artocarpus incisus, impatiens balsamina, alpinia officinarum, epilobium, magnolia, pueraria thumbergiana, scoparia dulcis, angelica gigas, pygeum africanum, tripterygium wilfordii and scutellaria baicalensis.

7. The method of claim 1 wherein the quantity administered comprises a daily dosage between 50-1000 mg per day.

8. The method of claim 7 wherein the quantity administered is 50 to 250 mg per day.

9. The method of claim 8 wherein the quantity administered is 100 mg per day.

10. The method of claim 9 wherein the quantity administered is in unit doses of 50 mg twice a day.

11. The method of claim 1 wherein the quantity is administered in the form of one of a gel capsule and a liquid suspension.
